(19) United States
(12) Patent Application Publication
(10) Pub. No.: US 2015/0057471 A1
(43) Pub. Date: Feb. 26, 2015

(54) CHLORINATING AGENTS

(71) Applicants: Max M Tirtowidjojo, Lake Jackson, TX (US); Matthew Lee Grandbois, Midland, MI (US); John D. Myers, Baton Rouge, LA (US); William J. Kruper, JR., Sanford, MI (US)

(72) Inventors: Max M Tirtowidjojo, Lake Jackson, TX (US); Matthew Lee Grandbois, Midland, MI (US); John D. Myers, Baton Rouge, LA (US); William J. Kruper, JR., Sanford, MI (US)

(73) Assignee: DOW GLOBAL TECHNOLOGIES LLC, Midland, MI (US)

(21) Appl. No.: 14/366,984

(22) PCT Filed: May 18, 2012
(86) PCT No.: PCT/US12/38634

§ 371 Date: Oct. 13, 2014

Related U.S. Application Data
(60) Provisional application No. 61/579,784, filed on Dec. 23, 2011.

(51) Int. Cl. C07C 17/013; C07C 17/25

(52) U.S. Cl. CPC C07C 17/013; C07C 17/25
USPC 570/155; 570/261

(57) ABSTRACT

The use of sulfuryl chloride, either alone or in combination with chlorine, as a chlorinating agent is disclosed.

### FIELD

The present invention relates to the use of sulfuryl chloride, either alone or in combination with chlorine, as a chlorinating agent.

### BACKGROUND

Hydrofluorocarbon (HFC) products are widely utilized in many applications, including refrigeration, air conditioning, foam expansion, and as propellants for aerosol products including medical aerosol devices. Although HFC's have proven to be more climate friendly than the chlorofluorocarbon and hydrochlorofluorocarbon products that they replaced, it has now been discovered that they exhibit an appreciable global warming potential (GWP).

The search for more acceptable alternatives to current fluorocarbon products has led to the emergence of hydrofluoroolefin (HFO) products. Relative to their predecessors, HFOs are expected to exert less impact on the atmosphere in the form of a lesser detrimental impact on the ozone layer and their generally lower GWP. Advantageously, HFO's also exhibit low flammability and low toxicity.

As the environmental, and thus, economic importance of HFO's has developed, so has the demand for precursors utilized in their production. Many desirable HFO compounds, e.g., such as 2,3,3,3-tetrafluoroprop-1-ene or 1,3,3,3-tetrafluoroprop-1-ene, may typically be produced utilizing feedstocks of chlorocarbons, and in particular, chlorinated propenes, which may also find use as feedstocks for the manufacture of polyurethane blowing agents, biocides and polymers.

Unfortunately, many chlorinated propenes may have limited commercial availability, and/or may only be available at prohibitively high cost, due at least in part to the fact that many conventional processes therefore utilize gaseous chlorine as a chlorinating agent. Because the chlorinating agent is in gaseous form, the concentration that may be achieved in liquid phase reactions is limited to the solubility of the gas therein. And, the mixing of gaseous reactants, chlorinating agents, solvents and/or catalysts may also be suboptimal. Typically, higher temperatures or pressures have been utilized to overcome these limitations, thereby adding undesirable time and/or cost to the process. For some manufacturers, the utilization of gaseous chlorine can represent transportation and safety issues.

It would thus be desirable to provide improved processes for the production of chlorocarbon precursors useful as feedstocks in the synthesis of refrigerants and other commercial products. More particularly, such processes would provide an improvement over the current state of the art if they made use of chlorinating agents available in a liquid form.

### BRIEF DESCRIPTION

The present invention provides such processes. More particularly, the present processes utilize sulfuryl chloride as a chlorinating agent for a feedstream comprising a saturated hydrocarbon and/or a saturated halogenated hydrocarbon. Unlike chlorine gas, sulfuryl chloride is a solvent and can act to increase the concentration of available chlorine in a liquid phase reaction. Furthermore, sulfuryl chloride can help dissolve catalysts that may desirably be utilized in such process, and as a result, acceptable reaction rates can be achieved without the application of excessive and/or expensive temperatures and pressures. In some embodiments, the selectivity to desired products can be improved. Indeed, because sulfuryl chloride is a liquid at temperatures lower than 70° C. and ambient pressure, it is less costly to mix with other reactants than gaseous chlorinating agents, such as chlorine.

In one aspect, there is provided a chemical manufacturing process comprising the use of SO₂Cl₂ as a chlorinating agent wherein a process feedstock comprises a saturated hydrocarbon. The process may be one for the manufacture of chlorinated propanes and/or propenes, and in some embodiments, those comprising 3-5 chlorine atoms. In some embodiments, the chlorinated propene produced may comprise 1,1,2,3-tetrachloropropene. The feedstock may comprise any feedstock desirably chlorinated, including, for example, propane, one or more dichloropropanes and/or one or more trichloropropanes.

The process comprises a at least one liquid phase chlorination step, which may desirably be conducted in the presence of a free radical initiator or an ionic chlorination catalyst. Suitable free radical initiators comprise AIBN, 2,2′-azobis(2,4-dimethyl valeronitrile, dimethyl 2,2′-azobis(2-methylpropionate), 1,1′-azobis(cyclohexane-1-carbonitrile) or 1,1′-azobis(cyclohexanecarbonitrile (ABCN), ultraviolet light or combinations of these, while suitable ionic chlorination catalysts comprise aluminum chloride (AlCl₃), iodine (I₂), ferric chloride (FeCl₃) and other iron containing compounds, iodine, sulfur, antimony pentachloride (SbCl₅), boron trichloride (BCl₃), lanthanum halides, metal triflates, or combinations of these The chlorination step may be conducted in the presence of a solvent, such as PDC, trichloropropane isomers, tetrachloropropane isomers, carbon tetrachloride or combinations of these. In some embodiments, HCl is generated by the process and desirably recovered therefrom as anhydrous HCl. Unreacted chlorine and the SO₂ byproduct may be converted back to SO₂Cl₂, if desired. Further, one or more reactants may be generated within or upstream of the process.

The process may further comprise at least one dehydrochlorination step that can be carried out in the presence of a chemical base, i.e., a caustic cracking step, or, can be carried out using a catalyst, such as one comprising iron. In some embodiments, a catalytic cracking step may be carried out using ferric chloride. The dehydrochlorination step may occur prior to a first chlorination step in some embodiments.

The advantages provided by the present processes may be carried forward by utilizing the chlorinated products produced thereby to produce further downstream products, such as, e.g., 2,3,3,3-tetrafluoroprop-1-ene or 1,3,3,3-tetrafluoroprop-1-ene.

### DESCRIPTION OF THE FIGURES

FIG. 1 shows a schematic representation of a process according to one embodiment;

FIG. 2 shows a schematic representation of a process according to a further embodiment;

FIG. 3 shows a schematic representation of a process according to a further embodiment; and

FIG. 4 shows a schematic representation of a process according to further embodiment.

### DETAILED DESCRIPTION

The present specification provides certain definitions and methods to better define the present invention and to guide those of ordinary skill in the art in the practice of the present invention. Provision, or lack of the provision, of a definition for a particular term or phrase is not meant to imply any particular importance, or lack thereof. Rather, and unless otherwise noted, terms are to be understood according to conventional usage by those of ordinary skill in the relevant art.

The terms “first”, “second”, and the like, as used herein do not denote any order, quantity, or importance, but rather are used to distinguish one element from another. Also, the terms “a” and “an” do not denote a limitation of quantity, but rather denote the presence of at least one of the referenced item, and the terms “front”, “back”, “bottom”, and/or “top”, unless otherwise noted, are merely used for convenience of description, and are not limited to any one position or spatial orientation.

If ranges are disclosed, the endpoints of all ranges directed to the same component or property are inclusive and independently combinable (e.g., ranges of “up to 25 wt. %, or, more specifically, 5 wt. % to 20 wt. %,” is inclusive of the endpoints and all intermediate values of the ranges of “5 wt. % to 25 wt. %,” etc.). As used herein, percent (%) conversion is meant to indicate change in molar or mass flow of reactant in a reactor in ratio to the incoming flow, while percent (%) selectivity means the change in molar flow rate of product in a reactor in ratio to the change of molar flow rate of a reactant.

Reference throughout the specification to “one embodiment” or “an embodiment” means that a particular feature, structure, or characteristic described in connection with an embodiment is included in at least one embodiment. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

In some instances, “PDC” may be used as an abbreviation for 1,2-dichloropropane, “TCP” may be used as an abbreviation for 1,2,3-trichloropropane and “TCPE” may be used as an abbreviation for 1,1,2,3-tetrachloropropene. The terms “cracking” and “dehydrochlorination” are used interchangeably to refer to the same type of reaction, i.e., one resulting in the creation of a double bond typically via the removal of a hydrogen and a chlorine atom from adjacent carbon atoms in chlorinated hydrocarbon reagents.

The present invention provides processes that utilize sulfuryl chloride as a chlorinating agent for a feedstream comprising a saturated hydrocarbon. Although the use of sulfuryl chloride as a chlorinating agent may be known in connection with processes involving feedstreams comprising unsaturated hydrocarbons, its use in connection with processes involving feedstreams comprising saturated hydrocarbons is not, nor is it expected. This is at least because the addition of chlorine atoms across a double bond involves a different chemistry, than does the addition of chlorine atoms to a saturated molecule.

Furthermore, unlike chlorine gas, sulfuryl chloride is a solvent and can act to increase the concentration of available chlorine in a liquid phase reaction. And, sulfuryl chloride can help dissolve catalysts that may be desirable in such process. As a result, acceptable reaction rates can be achieved without the application of excessive and/or expensive temperatures and pressures. Indeed, because sulfuryl chloride is a liquid at temperatures lower than 70° C. and ambient pressure, it is less costly to mix with other reactants than gaseous chlorinating agents, such as chlorine. In other words, not only is the use of sulfuryl chloride as a chlorinating agent in connection with the chlorination of saturated hydrocarbons unknown and unexpected over its prior uses as a chlorinating agent of unsaturated hydrocarbons, its use provides unexpected results and advantages in processes for the chlorinating of a feedstream comprising a saturated hydrocarbon as compared to chlorine.

It has also now been surprisingly discovered that the use of the combination of sulfuryl chloride with chlorine can provide even better results in processes for the chlorination of saturated hydrocarbons, e.g., conversion at low intensity conditions, product yield, selectivity, and/or lower byproduct formation, than the use of either alone. In some embodiments, the results of the use of such a combination may be synergistic.

The present method may be applied to any chemical process wherein a feedstream comprising a saturated hydrocarbon is desirably chlorinated. Chlorinated hydrocarbons or olefins having fewer than 10 carbon atoms, or less than 8 carbon atoms, or less than 6 carbon atoms, or having from 1-3 carbon atoms have wide commercial applicability, and efficient processes for their manufacture are welcome in the art, and in some embodiments, the present processes may be directed to their preparation. In other embodiments, the process may desirably be a process for the production of a chlorinated propene.

Any chlorinated propene may be produced using the present method, although those with 3-5 chlorine atoms may have greater commercial applicability, and production of the same may thus be preferred in some embodiments. In some embodiments, the process may be used in the production of 1,1,2,3-tetrachloropropene, which may be preferred as a feedstock for refrigerants, polymers, biocides, etc.

The saturated hydrocarbon utilized in the feedstream is not particularly limited, and will depend upon the product desirably produced. Typically, the saturated hydrocarbon may have the same number of carbon atoms as the desired product, while in other embodiments, the saturated hydrocarbon may have fewer carbon atoms than the desired product. In those embodiments wherein the process is utilized to produce a chlorinated hydrocarbon or olefin having 5 or fewer carbon atoms, saturated hydrocarbons having from 1 carbon atom to three carbon atoms may be utilized.

The saturated hydrocarbon may also be halogenated, and in some embodiments, may be chlorinated. For example, in those embodiments, wherein chlorinated propanes or propenes are produced, the saturated hydrocarbon may comprise propane, and/or one or more monochloropropanes, dichloropropanes, such as 1,2-dichloropropane, or trichloropropanes. In those embodiments wherein tetrachloromethane is produced, the saturated hydrocarbon may comprise one or more chlorinated methanes.

The saturated hydrocarbon may be utilized alone, or in combination with one or more reactants and/or solvents. In many chlorination processes, unreacted reactants and/or reaction byproducts may desirably be recycled within the process, and so the feedstream may additionally comprise them. Unsaturated hydrocarbons may also be present in the feedstream, and may either be part of the initial feed, or recycled from the process.

In some embodiments, the sulfuryl chloride may be regenerated and reused within the process. That is, the chlorination reaction between sulfuryl chloride and a feedstream comprising one or more saturated hydrocarbons may typically produce SO₂ as a byproduct, and this may either be disposed of, fed to a downstream process and used as a reactant, or used to regenerate sulfuryl chloride by reaction with chlorine. Reaction conditions to regenerate sulfuryl chloride from sulfur dioxide are generally known to those of ordinary skill in the art, and any known method of doing so may be used, with some preference given to those readily incorporated into the process, i.e., as by being capable of implementation in existing equipment and/or with existing reactants.

Catalysts are not required for the chlorination steps of the present process, but can be used, if desired, in order to increase the reaction kinetics. In some embodiments, known free radical catalysts or initiators are desirably used to enhance the present process. Such catalysts may typically comprise one or more chlorine, peroxide or azo-(R—N═N—R′) groups and/or exhibit reactor phase mobility/activity. As used herein, the phrase “reactor phase mobility/activity” means that a substantial amount of the catalyst or initiator is available for generating free radicals of sufficient energy which can initiate and propagate effective turnover of the product, the chlorinated and/or fluorinated propene(s), within the design limitations of the reactor.

Furthermore, if a free radical catalyst/initiator is used, the catalyst/initiator should have sufficient homolytic dissociation energies such that the theoretical maximum of free radicals is generated from a given initiator under the temperature/residence time of the process. It is especially useful to use free radical initiators at concentrations where free radical chlorination of incipient radicals is prevented due to low concentration or reactivity. Surprisingly, the utilization of the same, does not result in an increase in the production of impurities by the process, but does provide selectivities to the chlorinated propenes of at least 50%, or up to 60%, up to 70%, and in some embodiments, up to 80% or even higher.

Such free radical initiators are well known to those skilled in the art and have been reviewed, e.g., in “Aspects of some initiation and propagation processes,” Bamford, Clement H. Univ. Liverpool, Liverpool, UK., Pure and Applied Chemistry, (1967), 15(3-4), 333-48 and Sheppard, C. S.; Mageli, O. L. “Peroxides and peroxy compounds, organic,” Kirk-Othmer Encycl. Chem. Technol., 3rd Ed. (1982), 17, 27-90.

Taking the above into consideration, examples of suitable catalysts/initiators comprising chlorine include, but are not limited to carbon tetrachloride, hexachloroacetone, chloroform, hexachloroethane, phosgene, thionyl chloride, sulfuryl chloride, trichloromethylbenzene, perchlorinated alkylaryl functional groups, or organic and inorganic hypochlorites, including hypochlorous acid, and t-butylhypochlorite, methylhypochlorite, chlorinated amines (chloramine) and chlorinated amides or sulfonamides such as chloroamine-T®, and the like. Examples of suitable catalysts/initiators comprising one or more peroxide groups include hydrogen peroxide, hypochlorous acid, aliphatic and aromatic peroxides or hydroperoxides, including di-t-butyl peroxide, benzoyl peroxide, cumyl peroxide, benzoyl peroxide, methyl ethyl ketone peroxide, acetone peroxide and the like. Diperoxides offer an advantage of not being able to propagate competitive processes (e.g., the free radical chlorination of PDC to TCP (and its isomers) and tetrachloropropanes). In addition, compounds comprising one or more azo-groups (R—N═N—R′), such as azobisisobutyronitrile (AIBN), 2,2′-azobis(2,4-dimethyl valeronitrile, dimethyl 2,2′-azobis(2-methylpropionate), 1,1′-azobis(cyclohexane-1-carbonitrile) or 1,1′-azobis(cyclohexanecarbonitrile (ABCN), may have utility in effecting the chlorination of PDC to trichloropropanes and tetrachloropropanes under the conditions of this invention. Combinations of any of these may also be utilized.

The process or reactor zone may be subjected to pulse laser or continuous UV/visible light sources at a wavelength suitable for inducing photolysis of the free radical catalyst/initiator, as taught by Breslow, R. in Organic Reaction Mechanisms W. A. Benjamin Pub, New York p 223-224. Wavelengths from 300 to 700 nm of the light source are sufficient to dissociate commercially available radical initiators. Such light sources include, e.g., Hanovia UV discharge lamps, sunlamps or even pulsed laser beams of appropriate wavelength or energy which are configured to irradiate the reactor chamber. Alternatively, chloropropyl radicals may be generated from microwave discharge into a bromochloromethane feedsource introduced to the reactor as taught by Bailleux et al., in Journal of Molecular Spectroscopy, 2005, vol. 229, pp. 140-144.

In some embodiments, ionic chlorination catalysts may be utilized in one or more chlorination steps. The use of ionic chlorination catalysts in the present process is particularly advantageous since they dehydrochlorinate and chlorinate alkanes during the same reaction. That is, ionic chlorination catalysts remove a chlorine and hydrogen from adjacent carbon atoms, the adjacent carbon atoms form a double bond, and HCl is released. A chlorine molecule is then added back, replacing the double bond, to provide a higher chlorinated alkane.

Ionic chlorination catalysts are well known to those or ordinary art and any of these may be used in the present process. Suitable ionic chlorination catalysts include, but are not limited to, aluminum chloride (AlCl₃), iodine (I₂), ferric chloride (FeCl₃) and other iron containing compounds, iodine, sulfur, antimony pentachloride (SbCl₅), boron trichloride (BCl₃), lanthanum halides, metal triflates, or combinations of these. If ionic chlorination catalysts are to be utilized in one or more of the chlorination steps of the present process, the use of AlCl₃ with or without I₂, can be preferred.

In some embodiments, the dehydrochlorination steps of the present process may be carried out in the presence of a catalyst so that the reaction rate is enhanced and also use of liquid caustic is reduced, or even eliminated, from the process. Such embodiments are further advantageous in that anhydrous HCl is produced, which is a higher value byproduct than aqueous HCl. If the use of catalysts is desired, suitable dehydrochlorination catalysts include, but are not limited to, ferric chloride (FeCl₃) as a substitute to caustic.

In other embodiments, one or more of the dehydrochlorination steps of the present process may be conducted in the presence of a liquid caustic. Although vapor phase dehydrochlorinations advantageously result in the formation of a higher value byproduct than liquid phase dehydrochlorinations, liquid phase dehydrochlorination reactions can provide cost savings since evaporation of reactants is not required. The lower reaction temperatures used in liquid phase reactions may also result in lower fouling rates than the higher temperatures used in connection with gas phase reactions, and so reactor lifetimes may also be optimized when at least one liquid phase dehydrochlorination is utilized.

Many chemical bases are known in the art to be useful for liquid dehydrochlorinations, and any of these can be used. For example, suitable bases include, but are not limited to, alkali metal hydroxides, such as sodium hydroxide, potassium hydroxide, calcium hydroxide; alkali metal carbonates such as sodium carbonate; lithium, rubidium, and cesium or combinations of these. Phase transfer catalysts such as quaternary ammonium and quaternary phosphonium salts (e.g. benzyltrimethylammonium chloride or hexadecyltributylphosphonium bromide) can also be added to improve the dehydrohalogenation reaction rate with these chemical bases.

Any or all of the catalysts utilized in the process can be provided either in bulk or in connection with a substrate, such as activated carbon, graphite, silica, alumina, zeolites, fluorinated graphite and fluorinated alumina. Whatever the desired catalyst (if any), or format thereof, those of ordinary skill in the art are well aware of methods of determining the appropriate format and method of introduction thereof. For example, many catalysts are typically introduced into the reactor zone as a separate feed, or in solution with other reactants.

The amount of any catalyst utilized will depend upon the particular catalyst chosen as well as the other reaction conditions. Generally speaking, in those embodiments of the invention wherein the utilization of a catalyst is desired, enough of the catalyst should be utilized to provide some improvement to reaction process conditions (e.g., a reduction in required temperature) or realized products, but yet not be more than will provide any additional benefit, if only for reasons of economic practicality.

For purposes of illustration only, then, it is expected in those embodiments wherein an ionic chlorination catalyst, e.g., comprising AlCl₃ and/or I₂, or free radical catalyst, e.g., comprising AIBN, is used, that useful concentrations of each will range from 0.001% to 20% by weight, or from 0.01% to 10%, or from 0.1% to 5 wt. %, inclusive of all subranges therebetween. If a dehydrochlorination catalyst is utilized, useful concentrations may range from 0.01 wt. % to 5 wt. % or from 0.05 wt. % to 2 wt. % at temperatures of 70° C. to 200° C. If a chemical base is utilized for one or more dehydrochlorinations, useful concentrations of these will range from 0.01 to 20 grmole/L, or from 0.1 grmole/L to 15 grmole/L, or from 1 grmole/L to 10 grmole/L, inclusive of all subranges therebetween. Relative concentrations of each catalyst/base are given relative to the feed, e.g., 1,2-dichloropropane alone or in combination with 1,2,3-trichloropropane.

In additional embodiments, one or more reaction conditions of the process may be optimized, in order to provide even further advantages, i.e., improvements in selectivity, conversion or production of reaction by-products. In certain embodiments, multiple reaction conditions are optimized and even further improvements in selectivity, conversion and production of reaction by-products produced can be seen.

Reaction conditions of the process that may be optimized include any reaction condition conveniently adjusted, e.g., that may be adjusted via utilization of equipment and/or materials already present in the manufacturing footprint, or that may be obtained at low resource cost. Examples of such conditions may include, but are not limited to, adjustments to temperature, pressure, flow rates, molar ratios of reactants, mechanical mixing, etc.

That being said, the particular conditions employed at each step described herein are not critical, and are readily determined by those of ordinary skill in the art. What is important is that sulfuryl chloride is utilized as a chlorinating agent. Those of ordinary skill in the art will readily be able to determine suitable equipment for each step, as well as the particular conditions at which the distillation/fractionation, drying, chlorination, cracking and isomerization steps described herein are conducted.

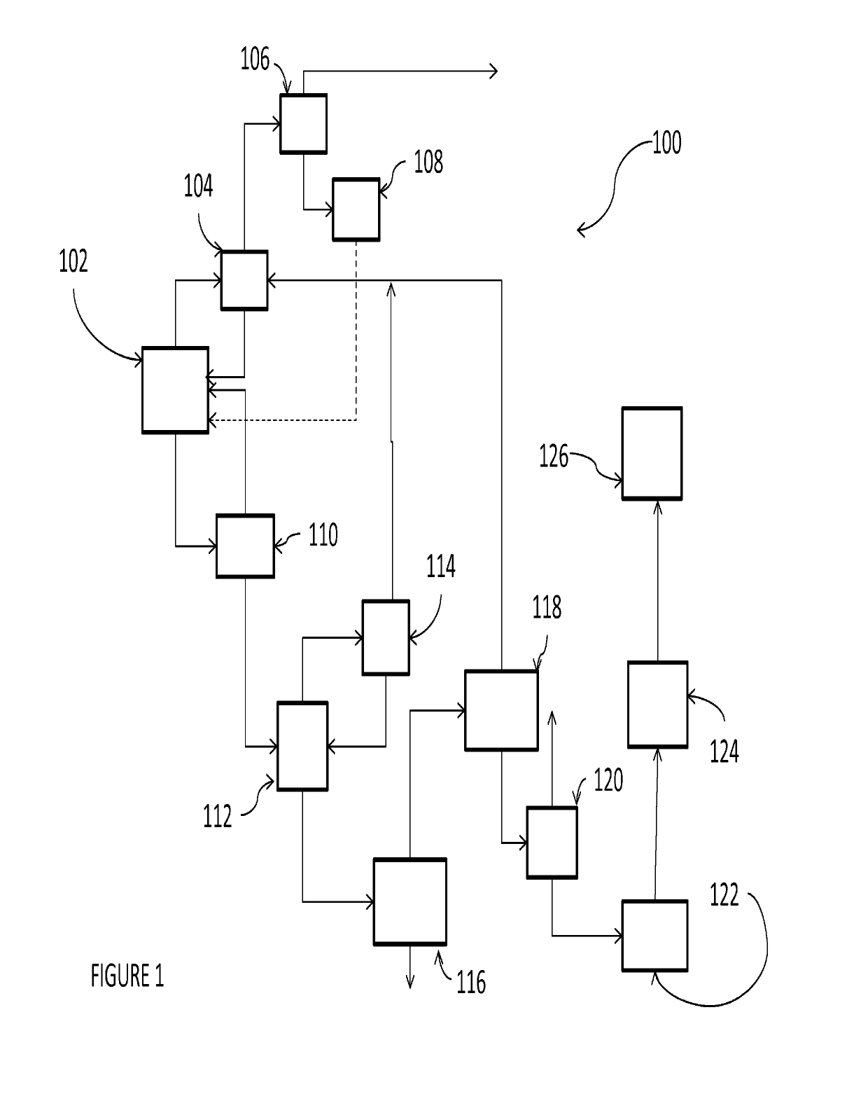

A schematic illustration of such a process is shown in FIG. 1. As shown in FIG. 1, process 100 would make use of chlorination reactors 102, 108 and 114, separation columns 104, 106, 110, 112, 116 and 120, dehydrochlorination reactors 118 and 122, drying column 124, and isomerization reactor 126. In operation, a feedstock comprising a saturated hydrocarbon, e.g., a dichloropropane, and SO₂Cl₂ is fed to chlorination reactor 102, which may be operated at any set of conditions operable to provide for the chlorination of PDC to tri-, tetra- and pentachlorinated propanes.

The overhead stream from chlorination reactor 102 comprises, HCl, unreacted monochloropropane, PDC, Cl₂ and SO₂, and excess SO₂Cl₂. After purifying and removing HClCl₂, and SO₂ in the overhead stream of separation column 104, the bottom stream, comprising mostly unreacted PDC and SO₂Cl₂, is recycled back to chlorination reactor 102. The overhead stream of column 104 comprising HCl, Cl₂, and SO₂, is send to separation column 106 where HCl is recovered in an overhead stream. The bottom stream of separation column 106 comprising Cl₂ and SO₂ is fed to chlorination reactor 108 and chlorinated with additional fresh Cl₂ to produce SO₂Cl₂, which may then be recycled back to chlorination reactor 102.

The bottom stream of chlorination reactor 102 is provided to separation column 110, which is operated at conditions effective to provide a bottoms stream comprising 1,1,2,3-tetrachloropropane, pentachloropropanes and heavier reaction by-products, and an overhead stream comprising TCP and other tetrachloropropane isomers. The overhead stream from separation column 110 is recycled to chlorination reactor 102, while the bottoms stream from separation column 110 is fed to separation column 112.

Separation column 112 separates 1,1,2,3-tetrachloropropane from pentachloropropane isomers and provides it as an overhead stream to chlorination reactor 114. Chlorination reactor 114 is desirably operated at conditions effective to maximize the production of the desirable pentachloropropane isomers, i.e., 1,1,1,2,3-pentachloropropane and 1,1,2,2,3-pentachloropropane, while minimizing the production of the less desirable 1,1,2,3,3 pentachloropropane isomer.

The bottom product stream from chlorination reactor 114, comprising unreacted 1,1,2,3-tetrachloropropane and the desired pentachloropropane isomers, is recycled to separation column 112. The overhead stream from chlorination reactor 114, comprising HCl and excess SO₂Cl₂ and/or Cl₂, is recycled to separation column 104. After purifying and removing HClCl₂, and SO₂ in the overhead stream of separation column 104, the bottom stream, comprising mostly unreacted PDC and SO₂Cl₂, is recycled back to chlorination reactor 102.

The bottoms stream from separation column 112 is fed to separation column 116, which is operated at conditions effective to provide an overhead stream comprising the desirable pentachloropropane isomers (1,1,2,2,3-pentachloropropane and 1,1,1,2,3-pentachloropropane) and a bottom stream comprising the less desirable 1,1,2,3,3-pentachloropropane, hexachloropropane and heavier by-products. The overhead stream from separation column 116 is fed to catalytic dehydrochlorination reactor 118, while the bottoms stream is appropriately disposed of.

Within dehydrochlorination reactor 118, the desirable pentachloropropane isomers are catalytically dehydrochlorinated to provide 1,1,2,3-tetrachloropropene. More specifically, dehydrochlorination reactor 118 may be charged with, e.g., iron or an iron containing catalyst such as FeCl₃ and operated at pressures of from ambient to 400 kPA, at temperatures of from 40° C. to 150° C. and with a residence time of less than 3 hours.

The bottom reaction stream from dehydrochlorination reactor 118 is provided to separation column 120, while the overhead stream from dehydrochlorination reactor 118 is provided to separation column 104 for further purification and recovery of anhydrous HCl, as described above.

Separation column 120 is operated at conditions effective to separate the desired chlorinated propene, e.g., 1,1,2,3-TCPE, as an overhead stream from the remaining by-products, e.g., 1,1,2,2,3-pentachloropropane. The bottoms stream from separation column 120 is fed to caustic dehydrochlorination reactor 122, and the product stream thereof provided to drying column 124, and then to isomerization reactor 126 to isomerize the 2,3,3,3-tetrachloropropene to 1,1,2,3-tetrachloropropene under the appropriate conditions.

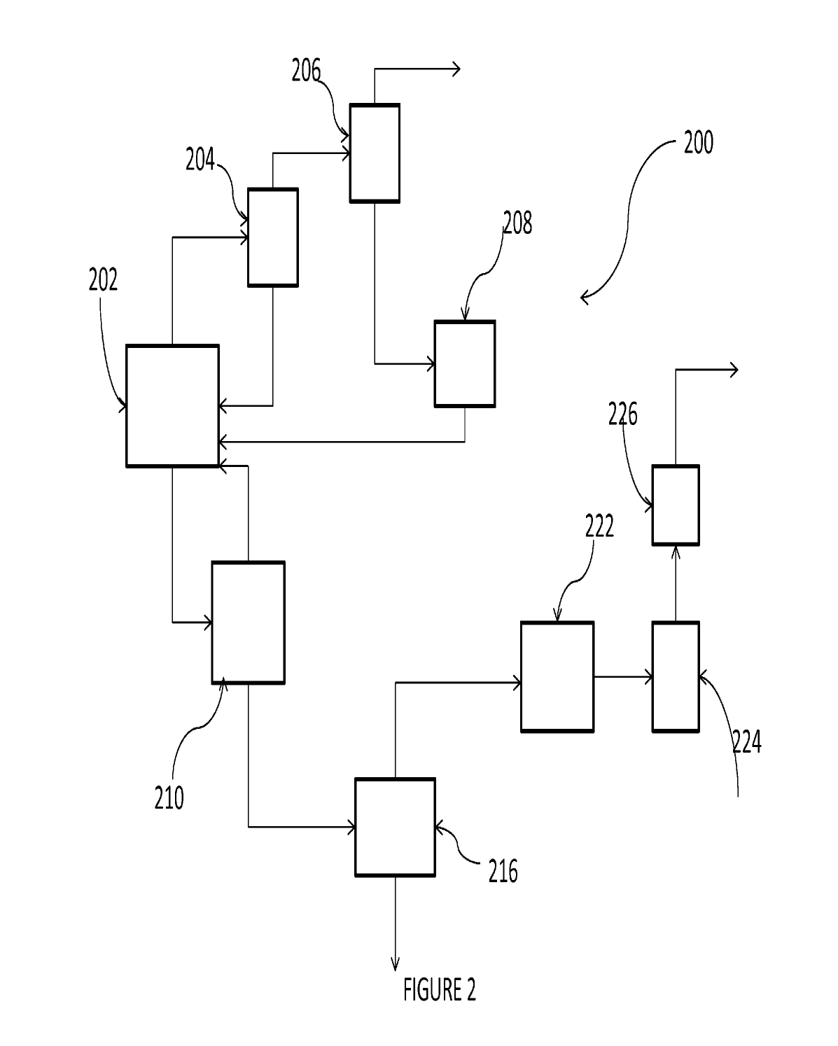

Another embodiment of the process is shown in FIG. 2. As shown, process 200 would make use of chlorination reactors 202 and 208, HCl recovery column 206, separation columns 204, 210 and 216, dehydrochlorination reactor 222, drying column 224 and isomerization reactor 226. In operation, a saturated hydrocarbon, e.g., 1,2-dichloropropane (alone or in combination with trichloropropane), SO₂Cl₂, and one or more free radical initiators such as AIBN are fed to chlorination reactor 202, which may be operated at any set of conditions operable to provide for the chlorination of PDC to tri-, tetra- and pentachlorinated propanes. In some embodiments, reactor 202 may be operated at conditions effective to provide a selectivity to 1,1,2,3,3-pentachloropropane of less than 5%, as described above.

The vapor overhead of chlorination reactor 202 comprises SO₂, Cl₂, HCl byproducts and some unreacted SO₂Cl₂ and PDC. After purifying and removing HCl, Cl₂, and SO₂ in the overhead stream of separation column 204, the bottom stream, comprising mostly unreacted PDC and SO₂Cl₂, is recycled back to reactor 202. The overhead stream of separation column 204, comprising HCl, Cl₂, and SO₂, is sent to HCl recovery column 206 where HCl is recovered in the overhead stream.

The bottom stream of HCl recovery column 206, comprising Cl₂ and SO₂, is fed to chlorination reactor 208 and chlorinated with additional fresh Cl₂ to produce SO₂Cl₂, which may then be recycled back to chlorination reactor 202.

The bottom stream of reactor 202 is fed to separation column 210, which is operated at conditions effective to separate the tri- and tetrachlorinated propanes from the pentachlorinated propanes. The tri- and tetrachlorinated propanes are recycled back to chlorination reactor 202 for further conversion/chlorination, while the bottom stream from separation column 210 is fed to separation column 216.

Separation column 216 separates the bottom stream from separation column 210 into an overhead stream comprising the desirable pentachloropropane isomers (1,1,1,2,2-pentachloropropane, 1,1,2,2,3-pentachloropropane and 1,1,1,2,3-pentachloropropane) and a bottom stream comprising the less desirable 1,1,2,3,3-pentachloropropane, hexachloropropane and heavier by-products. The overhead stream from separation column 216 is fed to dehydrochlorination reactor 222, while the bottoms stream from separation column 216 is appropriately disposed of.

Within dehydrochlorination reactor 222, the desirable pentachloropropane isomers are caustic cracked using sodium hydroxide to provide 2,3,3,3-tetrachloroproene and 1,1,2,3-tetrachloropropene. The product stream of dehydrochlorination reactor 222 is fed to drying column 224, and then to isomerization reactor 226, wherein the dried 2,3,3,3-tetrachloropropene is isomerized to TCPE.

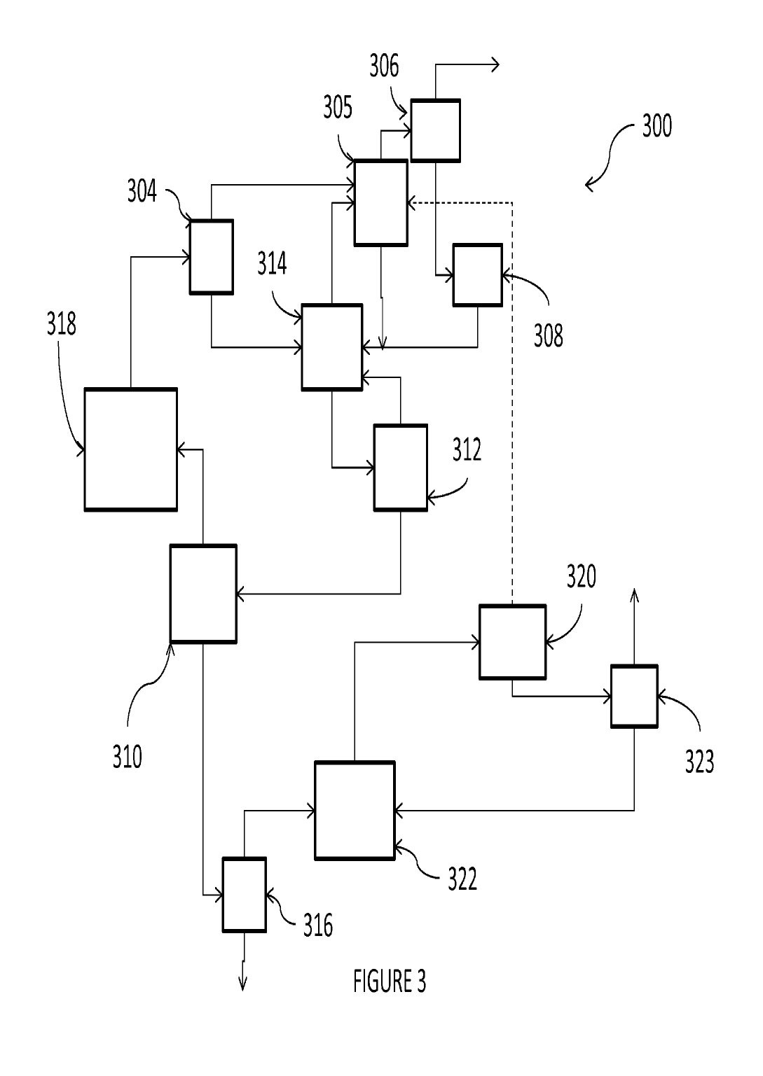

Yet another embodiment of the process is shown in FIG. 3. As shown, process 300 would make use of vapor phase dehydrochlorination reactors 318 and 322, separation columns 304, 305, 306, 310, 312, 316, 320 and 323 and chlorination reactors 308 and 314. In operation, 1,2,3-trichloropropane and recycled tetrachloropropane are fed into dehydrochlorination reactor 318, which is desirably operated at conditions sufficient to produce HCl, and 2,3-dichloropropene, 1,2,3-trichloropropene and unreacted chlorinated propanes.

The reaction stream from dehydrochlorination reactor 318 is fed to separation column 304 for the removal of lights and HCl in the overhead stream. The overhead stream from separation column 304 is fed to separation column 305 for further purification of HCl and recovery of 2,3-dichloropropene, and/or dichloropropene intermediates.

The bottoms stream from separation column 304 comprising 2,3-dichloropropene, 1,2,3-trichloropropene and unreacted TCP and tetrachloropropanes is fed to chlorination reactor 314, which is fed with sulfuryl chloride and produces a bottom stream comprising 1,2,2,3-tetrachloropropane and 1,1,2,2,3 pentachloropropane.

The overhead stream produced by chlorination reactor 314, comprising SO₂, Cl₂, HCl and a small fraction of SO₂Cl₂, is fed to a separation column 305, which is operated at conditions effective to provide excess SO₂Cl₂ and unreacted 2,3-dichloropropene in a bottom stream which is then recycled to chlorination reactor 314.

The overhead stream from separation column 305, comprising HCl, SO₂, and Cl₂, is fed to HCl recovery column 306 to purify HCl in an overhead stream. The bottom stream of HCl recovery column 306, comprising SO₂ and Cl₂ is fed to chlorination reactor 308 with fresh Cl₂ to produce SO₂Cl₂ which is recycled to chlorination reactor 314. The bottom stream of chlorination reactor 314, comprising 1,2,2,3-tetrachloropropane, 1,1,2,2,3-pentachloropropane, 2,3-dichloropropene and unreacted SO₂Cl₂, is fed to separation column 312.

The overhead stream from separation column 312, comprising SO₂Cl₂ and 2,3-dichloropropene, is recycled back to chlorination reactor 314. The bottom stream from separation column 314, comprising TCP, and tetrachloropropane and pentachloropropane intermediates, is fed to separation column 310.

1,2,3 TCP and 1,2,2,3 tetrachloropropane are recovered by separation column 310 in an overhead stream and recycled to dehydrochlorination reactor 318. 1,1,2,2,3 pentachloropropane is provided as a bottoms stream from separation column 310 and fed to separation column 316. Separation column 316 is operated at conditions effective to provide pentachloropropanes in an overhead stream, and heavier byproducts in a bottom stream.

The overhead stream from separation column 316 is sent to dehydrochlorination reactor 322, which produces an overhead stream comprising 1,1,2,3-TCPE. Additional HCl may be recovered from this product stream by providing it to separation column 320 (optional). The bottom stream from separation column 320, comprising the desired 1,1,2,3-TCPE and unreacted pentachloropropane, may be provided to separation column 323, which can provide purified TCPE in an overhead stream, and a bottom stream comprising unreacted pentachloropropane, which may be recycled to dehydrochlorination reactor 322.

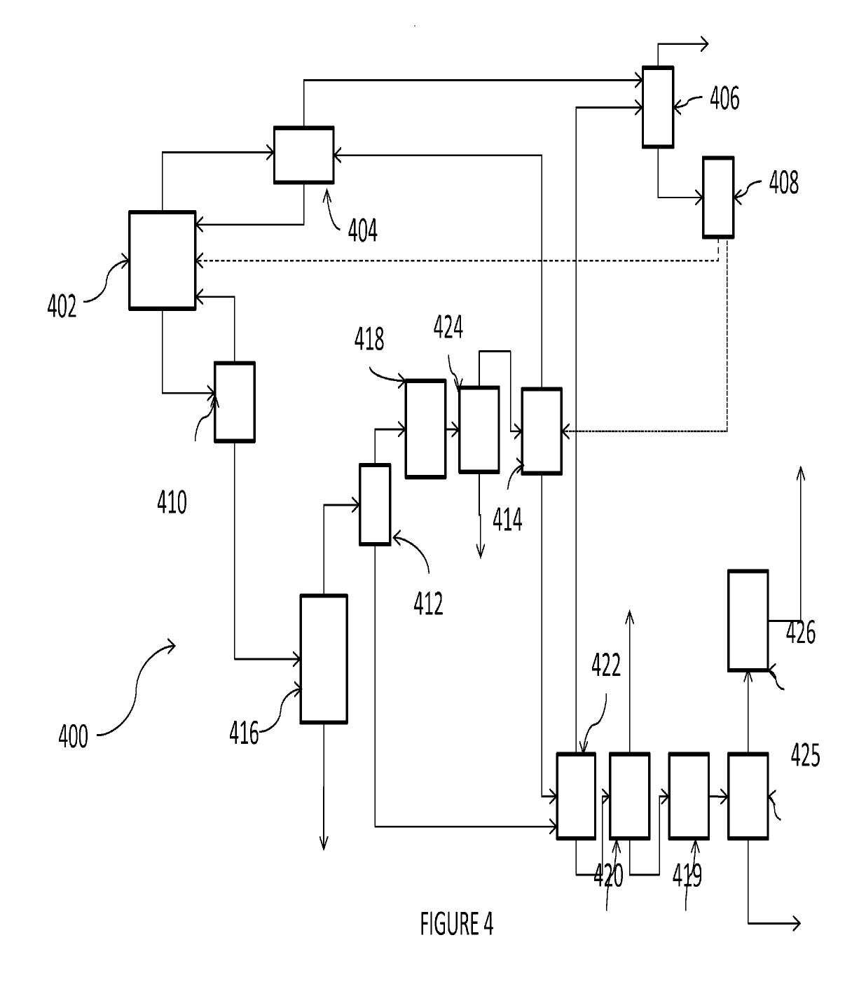

Yet another embodiment of the process is schematically illustrated in FIG. 4. As shown in FIG. 4, process 400 would make use of chlorination reactors 402, 408 and 414, separation columns 404, 406, 410, 412, and 416, dehydrochlorination reactors 418, 419 and 422, drying columns 424 and 425 and isomerization reactor 426.

In operation, 1,2,3-trichloropropane (alone or, in some embodiments, in combination with recycled 1,2,2,3-tetrachloropropane) and SO₂Cl₂ are fed to chlorination reactor 402, which may be operated at any set of conditions operable to provide for the chlorination of TCP to tetra- and pentachlorinated propanes and known to those of ordinary skill in the art. The overhead stream of chlorination reactor 402 is fed to separation column 404, which may desirably be a distillation column. The column is operated such that the overhead stream therefrom comprises SO₂, Cl₂ and HCl. The bottom stream of column 404 comprising unreacted SO₂Cl₂ and TCP may be recycled to chlorination reactor 402.

The overhead stream from separation column 404 is desirably condensed and provided to separation column 406 for the recovery of anhydrous HCl in an overhead stream thereof. The bottom stream from separation column 406, comprising chlorine and SO₂, is fed to chlorination reactor 408 with fresh Cl₂ to regenerate SO₂Cl₂ that may then be recycled to chlorination reactor(s) 402 and/or 414.

The bottom stream of reactor 402 is fed to separation column 410, which is operated at conditions effective to provide an overhead stream comprising TCP and 1,2,2,3-tetrachloropropane and a bottoms stream comprising other tetrachloropropane isomers, pentachloropropanes and heavier reaction by-products. The overhead stream from separation column 410 may be recycled to chlorination reactor 402, while the bottoms stream from separation column 406 is fed to separation column 416.

Separation column 416 separates the bottom stream from column 410 into an overhead stream comprising 1,1,2,3-tetrachloropropane, the desirable pentachloropropane isomers (1,1,2,2,3-pentachloropropane and 1,1,1,2,3-pentachloropropane) and a bottom stream comprising the less desirable 1,1,2,3,3-pentachloropropane, hexachloropropane and heavier by-products. The overhead stream from separation column 416 is fed to separation column 412, while the bottoms stream is appropriately disposed of.

Separation column 412 separates the overhead stream from separation column 416 into an overhead stream comprising 1,1,2,3-tetrachloropropane and a bottoms stream comprising desired pentachloropropanes isomers, e.g., 1,1,2,2,3 and 1,1,1,2,3-pentachloropropane. The 1,1,2,3-tetrachloropropane is then caustic cracked in dehydrochlorination reactor 418 to provide trichloropropene intermediates.

The reaction liquid from dehydrochlorination reactor 418 is fed to drying column 424 and the dried stream fed to chlorination reactor 414. Excess SO₂Cl₂, chlorine and SO₂ from chlorination reactor 414 may be recycled to separation column 404, if desired. The product stream from chlorination reactor 414, expected to comprise 1,1,2,2,3 and 1,1,1,2,3-pentachloropropane, is fed to dehydrochlorination reactor 422, where it is combined with the bottoms stream from separation column 412 that also comprises 1,1,2,2,3- and 1,1,1,2,3-pentachloropropane.

Within dehydrochlorination reactor 422, the desirable pentachloropropane isomers are catalytically dehydrochlorinated to provide 1,1,2,3-tetrachloropropene. The bottom reaction stream from dehydrochlorination reactor 422 is fed to separation column 420, while the overhead stream, comprising anhydrous HCl, is provided to separation column 406 for purification and recovery of anhydrous HCl.

Separation column 420 is operated at conditions effective to separate the desired chlorinated propene, e.g., 1,1,2,3-TCPE, as an overhead stream from the remaining by-products, e.g., 1,1,2,2,3-pentachloropropane. The bottoms stream from separation column 420 is fed to caustic dehydrochlorination reactor 419, and the product stream thereof provided to drying column 424. The dried stream from drying column 424 is provided to isomerization reactor 426 to isomerize the 2,3,3,3-tetrachloropropene to 1,1,2,3-tetrachloropropene under the appropriate conditions.

The chlorinated propenes produced by the present process may typically be processed to provide further downstream products including hydrofluoroolefins, such as, for example, 1,3,3,3-tetrafluoroprop-1-ene (HFO-1234ze). Since the present invention provides an improved process for the production of chlorinated propenes, it is contemplated that the improvements provided will carry forward to provide improvements to these downstream processes and/or products. Improved methods for the production of hydrofluoroolefins, e.g., such as 2,3,3,3-tetrafluoroprop-1-ene (HFO-1234yf), are thus also provided herein.

The conversion of chlorinated propenes to provide hydrofluoroolefins may broadly comprise a single reaction or two or more reactions involving fluorination of a compound of the formula C(X)ₘCCl(Y)ₙ(C)(X)ₘ to at least one compound of the formula CF₃CF═CHZ, where each X, Y and Z is independently H, F, Cl, I or Br, and each m is independently 1, 2 or 3 and n is 0 or 1. A more specific example might involve a multi-step process wherein a feedstock of a chlorinated propene is fluorinated in a catalyzed, gas phase reaction to form a compound such as 1-chloro-3,3,3-trifluoropropene (1233zd). The 1-chloro-2,3,3,3-tetrafluoropropane is then dehydrochlorinated to 2,3,3,3-tetrafluoroprop-1-ene or 1,3,3,3-tetrafluoroprop-1-ene via a catalyzed, gas phase reaction.

Some embodiments of the invention will now be described in detail in the following examples.

### Example 1—Comparative

A 50 ml flask equipped with a magnetic stir bar, reflux condenser, mineral oil bubbler, and heating mantle is charged with 1,2-dichloropropane (5.79 g, 51.2 mmol), aluminum chloride (0.7 g, 5.2 mmol) and carbon tetrachloride (15.87 g, 10 mL) under an inert atmosphere. The mixture is heated to an internal temperature of 60° C. and then charged with chlorine (4.1 g, 57.8 mmol).

After 60 minutes, an aliquot of the reaction mixture is removed, quenched with water, and then extracted with methylene chloride prior to gas chromatographic analysis. The GC analysis shows a 8:1 112TCP to TCP product distribution with 75% conversion of PDC after 1 hour run time.

### Example 2—Inventive

A 50 ml flask equipped with a magnetic stir bar, reflux condenser, mineral oil bubbler, and heating mantle is charged with aluminum chloride (0.5 g, 3.7 mmol) and sulfuryl chloride (17 g, 126.0 mmol) under an inert atmosphere. The mixture is heated to an internal temperature of 60° C. and then charged with 1,2-dichloropropane (4.05 g, 35.9 mmol), which induces a rapid evolution of gas and a color change of the reaction mixture.

After 60 minutes, an aliquot of the reaction mixture is removed, quenched with water, and then extracted with methylene chloride prior to gas chromatographic analysis. The GC analysis shows an internal reaction speciation of 65% 1,2-dichloropropane, 33% 1,1,2-trichloropropane, 1% 1,2,3-trichloropropane, <0.5% 1,1,2,3-tetrachloropropane, <0.5% heavies. This shows that 35% conversion of PDC is observed with 33:1 molar ratio of 1,1,2-trichloropropane (112TCP) to 1,2,3-trichloropropane.

While the conversion in the comparative example using Cl₂ is higher, the overall yield to trichloropropane products is only 22% with Cl₂/CCl₄. In contrast, the overall yield to trichloropropane products is 31% using SO₂Cl₂.

### Example 3—Inventive

A 50 ml reactor equipped with an overhead agitator and heating mantle is charged with aluminum chloride (0.5 g, 3.7 mmol), sulfuryl chloride (17 g, 126.0 mmol), and chlorine (4.05 g, 35.9 mmol) under an inert atmosphere. The mixture is heated to an internal temperature of 60° C. and then charged with 1,2-dichloropropane (4.05 g, 35.9 mmol), which induces a rapid evolution of gas and a color change of the reaction mixture.

After 60 minutes, an aliquot of the reaction mixture is removed, quenched with water, and then extracted with methylene chloride prior to gas chromatographic analysis. The GC analysis shows a higher conversion of PDC and higher overall yield of trichloropropanes than example 1, along with a high regioselectivity towards 112TCP similar to example 2.

### Example 4—Inventive

This example illustrates the use of SO₂Cl₂ as chlorinating agent and the ionic chlorination catalysts I₂ and AlCl₃ to convert 1,2-dichloropropane to C₃H₅Cl₃, C₃H₄Cl₄, and C₃H₃Cl₅ isomers.

Chlorination of 0.95 gr of PDC to 1,1,2,2,3-pentachloropropane (240aa) is conducted with 4.5 molar equivalent of SO₂Cl₂ for 8 hours at from 50° C. to 70° C. A 4 dram vial equipped with micro-flea stir bar and water condenser at the overhead padded with N₂ is used. The combined catalysts (7 mg I₂, 20 mg AlCl₃) are added to the solvent under N₂ and the reaction is heated to 55° C. for 3 hours. The loss of HCl and SO₂ decreased over this period and so the reaction is heated to reflux (70° C. headspace) for 4 hours while monitoring by NMR. At 7 hours another 1 equivalent of SO₂Cl₂ (1.13 g) is added and reflux is continued for 1 more hour. The reaction content is then added to 5 mL cold water with mixing to give a clear white phase of oil. The bottom phase is carefully pipetted and the aqueous phase extracted with 4 mL of CH₂Cl₂. The combined organic phase is dried over MgSO₄ and evaporated to give 1.55 g (estimated 89% theoretical recovery) of a 4:1 ratio of mainly 1,1,2,2,3-PCP to 1,2,3-TCP.

The product molar distribution of the first 7 hr reaction with 3.5 molar ratio of SO₂Cl₂ to PDC is show in Table 1. The absence of 1,1,2,3,3-pentachloropropane (11233) is highly desirable as dehydrochlorination of the same can result in undesirable TCPE isomers (cis/trans-1,2,3,3-tetrachloropropenes and/or 1,1,3,3-tetrachloropropenes). On the other hand, dehydrochlorination of 1,1,2,2,3-pentachloropropane will result in either TCPE or 2,3,3,3-tetrachloropropene that is readily be isomerized to TCPE (See, e.g., U.S. Pat. No. 3,823,195). Dehydrochlorination of 1,1,1,2,2-pentatchloropropane results in desirable intermediate 2,3,3,3-tetrachloropropene. About 4.24% of the product is a mixture of hexachloropropanes, a waste intermediate. This amount can be minimized by adjusting the ratio of catalyst to reactant (i.e., SO₂Cl₂/PDC), reaction time, and/or temperature. The tri- and tetrachlorinated propane intermediates can also be recycled to improve the process yield.

TABLE 1

| | |
|---|---|
| 1,1,2,2,3-pentachloropropane | 53.05% |
| 1,1,2,3,3-pentachloropropane | 0.00% |
| 1,1,1,2,2-pentachloropropane | 1.33% |
| 1,1,1,2,3-pentachloropropane | 0.00% |
| 1,1,2,2-tetrachloropropane | 1.06% |
| 1,1,2,3-tetrachloropropane | 3.18% |
| 1,2,2,3-tetrachloropropane | 5.84% |
| 1,1,1,2-tetrachloropropane | 0.00% |
| 1,1,2-trichloropropane | 12.20% |
| 1,2,2-trichloropropane | 0.00 |
| 1,2,3-trichloropropane | 19.10% |
| Hexachloropropane isomers | 4.24% |

The product composition of further chlorination of reaction mixture shown in Table 1 using an additional 1 equimolar of SO₂Cl₂ is listed in Table 2. These results show that further chlorination of tri- and tetra-chlorinated propane intermediates leads to the desired 1,1,2,2,3-pentachloropropane and 1,1,1,2,2-pentachloropropane without substantial, or any, formation of 1,1,2,3,3-pentachloropropane.

TABLE 2

| | |
|---|---|
| 1,1,2,2,3-pentachloropropane | 66.36% |
| 1,1,2,3,3-pentachloropropane | 0.00% |
| 1,1,1,2,2-pentachloropropane | 0.46% |
| 1,1,1,2,3-pentachloropropane | 0.00% |
| 1,1,2,2-tetrachloropropane | 0.00% |
| 1,1,2,3-tetrachloropropane | 3.94% |
| 1,2,2,3-tetrachloropropane | 0.99% |
| 1,1,1,2-tetrachloropropane | 0.00% |
| 1,1,2-trichloropropane | 1.31% |
| 1,2,2-trichloropropane | 0.00% |
| 1,2,3-trichloropropane | 18.4% |
| Hexachloropropane isomers | 8.54% |

### Example 5

The use of SO₂Cl₂ as chlorinating agent and the free radical catalyst AIBN to convert 1,2-dichloropropane to C₃H₅Cl₃, C₃H₄Cl₄, and C₃H₃Cl₅ isomers.

In this example, liquid SO₂Cl₂ and PDC (1,2-dichloropropane) are mixed in a 100 ml flask heated in a water bath to maintain temperature 55° C. to 60° C. A reflux column is placed to return unreacted reactants that are stripped by SO₂ and HCl byproducts to the reaction. GC/MS is used to determine the product composition.

Table 1 shows the chlorinated C3 product distribution at various SO₂Cl₂ and AIBN initiator concentration at near complete PDC conversion. As also shown in FIG. 1, less than 8% molar selectivity to the less desirable byproduct 1,1,2,3,3-pentachloropropane (11233) is obtained at high excess SO₂Cl₂ at 45% conversion to pentachloropropane (C₃Cl₅) isomers. This shows that a process with selectivity >90% can be achieved when conversion to C₃Cl₅ is kept below 40% and partial chlorination of 1,1,2,3-tetrachloropropane is kept such that 11233 production is minimized by recycling of C₃H₅Cl₃ and C₃H₄Cl₄ intermediates.

SO2Cl2/PDC

| | | | | |
|---|---|---|---|---|
| 3 | 3 | 5 | 5 | 6 |

AIBN/PDC

| | | | | |
|---|---|---|---|---|
| 0 | 2 | 1 | 2 | 3 |

PDC conversion

| | | | | |
|---|---|---|---|---|
| 98.5% | 100.0% | 100.0% | 100.0% | 100.0% |

Selectivity

| | | | | | |
|---|---|---|---|---|---|
| 11223 | 3.3% | 3.7% | 5.0% | 11.8% | 19.4% |
| 11233 | 2.0% | 2.0% | 2.4% | 5.2% | 7.4% |
| 11122 | 1.3% | 1.7% | 2.5% | 6.3% | 10.7% |
| 11123 | 2.3% | 2.6% | 1.7% | 4.1% | 5.8% |
| 1122 | 13.2% | 17.8% | 19.4% | 21.2% | 23.9% |
| 1123 | 15.6% | 15.6% | 14.8% | 10.8% | 8.9% |
| 1223 | 10.1% | 11.8% | 12.3% | 12.9% | 9.7% |
| 1112 | 3.6% | 3.3% | 3.0% | 7.0% | 1.8% |
| 112 | 8.9% | 6.5% | 6.7% | 4.6% | 0.2% |
| 122 | 18.0% | 19.7% | 19.7% | 9.4% | 6.2% |
| 123 | 20.3% | 14.8% | 12.2% | 6.6% | 5.8% |

1. A chemical manufacturing process comprising the use of SO₂Cl₂ as a chlorinating agent in at least one chlorination step and further in the presence of no catalyst, an ionic chlorination catalyst or a free radical initiator, wherein a process feedstock comprises a saturated hydrocarbon having from 1 to 3 carbon atoms and/or a saturated halogenated hydrocarbon having from 1 to 3 carbon atoms and wherein, when the chlorination step is conducted in the presence of a free radical initiator, the free radical initiator is selected from the group consisting of UV/visible light and/or initiators comprising one or more chlorine or azo-groups.

2. The process of claim 1, wherein the process comprises one for the manufacture of chlorinated propanes and/or propenes.

3. The process of claim 2, wherein the chlorinated propane and/or propene comprises 3-5 chlorine atoms.

4. The process of claim 1, wherein the process feedstock comprises propane and/or one or more monochloropropanes.

5. The process of claim 1, wherein the process feedstock comprises a dichloropropane.

6. The process of claim 1, wherein the at least one chlorination step is conducted in the presence of a free radical initiator or an ionic chlorination catalyst.

7. The process of claim 6, wherein the free radical initiator comprises azobisisobutyronitrile, 2,2′-azobis(2,4-dimethyl valeronitrile, dimethyl 2,2′-azobis(2-methylpropionate), 1,1′-azobis(cyclohexane-1-carbonitrile) or 1,1′-azobis(cyclohexanecarbonitrile), ultraviolet light or combinations of these.

8. The process of claim 6, wherein the ionic chlorination catalyst comprises AlCl₃, I₂, FeCl₃, sulphur, iron, or combinations of these.

9. The process of claim 6, further comprising the use of a solvent in the chlorination step, wherein the solvent comprises 1,2-dichloropropane, trichloropropane isomers, tetrachloropropane isomers, carbon tetrachloride or combinations of these.

10. The process of claim 6, wherein at least one chlorination step generates a stream comprising unreacted SO₂Cl₂, Cl₂, SO₂ and HCl and the HCl is separated from the stream as anhydrous HCl.

11. The process of claim 1, wherein the process further comprises at least one dehydrochlorination step.

12. The process of claim 11, wherein the dehydrochlorination is carried out in the presence of at least one chemical base.

13. The process of claim 12, wherein the chemical base comprises NaOH, KOH, and or Ca(OH)₂.

14. The process of claim 1, wherein at least one component of the feedstock is generated within, or upstream of, the process.

15. A process for preparing 2,3,3,3-tetrafluoroprop-1-ene or 1,3,3,3-tetrafluoroprop-1-ene comprising converting a chlorinated propene and/or propane prepared by the process of claim 2 into 2,3,3,3-tetrafluoroprop-1-ene or 1,3,3,3-tetrafluoroprop-1-ene.
